(19) United States
(12) Patent Application Publication
(10) Pub. No.: US 2018/0098822 A1
(43) Pub. Date: Apr. 12, 2018

(54) ATTACHMENT OF AN IDENTIFICATION TAG

(71) Applicant: Caretag Surgical ApS, Hörsholm (DK)

(72) Inventor: Søren Bilsøe, Espergærde (DK)

(21) Appl. No.: 15/566,935

(22) PCT Filed: Apr. 15, 2016
(86) PCT No.: PCT/EP2016/058346

§ 371 Date: Oct. 16, 2017

(30) Foreign Application Priority Data
Apr. 17, 2015 (EP) 15164025.7

(51) Int. Cl. A61B 90/98

(52) U.S. Cl. CPC A61B 90/98; A61B 2017/00951

(57) ABSTRACT

A method and a medical instrument are disclosed. The medical instrument has a first part comprising an attachment section and a second part comprising one or more functional sections. The medical instrument comprises an identification tag being positioned within the attachment section, and a cover material forming a tube body having at least two openings including a first opening and a second opening. The cover material encloses the identification tag and the attachment section. The first part extends through the first opening and the second opening. The first opening and the second opening are occluded by the first part.

The present disclosure relates to a medical instrument, such as a surgical instrument, with an attached identification tag, such as an RFID tag, and a related method for attaching an identification tag to a medical instrument. Especially, an identification tag may be used for registration before and after an operation, or before and after sterilization of the medical instrument.

### BACKGROUND

In an operating room, certain type and a certain number of medical instruments may be needed to perform a procedure, such as a surgical procedure. Typically the medical instruments brought into the operating room prior to a procedure are counted and registered manually, to be certain that the right amount and correct instruments are available for the procedure. After completion of the procedure, the instruments are again counted and registered manually, to ensure that no instruments are missing.

Manually registering and counting of equipment, before and after a procedure, is both time consuming and ineffective. Further, errors may potentially result in a life threatening situation for the patient.

Other situations exist where medical instruments need to be registered, and where a more efficient procedure will be beneficial, e.g. before and after sterilization, in or out of maintenance etc.

Providing the available instruments with an identification tag, such as an electronic identification tag, such as an RFID tag or an NFC tag, it is possible to uniquely and automatically detect each individual instrument.

However, instruments are seldom manufactured with identification tags and several problems occur when attaching an identification tag to an instrument.

The identification tag may provide undue protrusions or sharp edges. This is especially a problem in surgical instruments for use in an operating room, the sharp edges may cause rubber gloves to be torn and pose a hygiene risk, both for the surgeon and the patient.

Further, cleaning, washing, and sterilization of the instruments may cause the identification tag to be separated from the instrument. Especially, an identification tag may be in risk of impacts by other instruments during washing, causing separation from the instrument and/or damage of the identification tag. Furthermore, sharp edges or grooves found on the identification tag and/or in the interface between the identification tag and the instrument may be difficult to clean.

Furthermore, medical instruments may be provided with surfaces complicating attachment of an identification tag. At least, the shape of the medical instrument may complicate attachment of an identification tag in a manner not subject to one or more of the above difficulties. For example using an adhesive, such as glue to attach the identification tag to a small surface area may not provide for a sufficiently strong fixation, resulting in unintentional dismounting of the identification tag.

### SUMMARY

It is an object of the present disclosure to provide medical instrument and a method for attaching an identification tag to a medical instrument, which overcomes at least some of the disadvantages of the prior art.

The present disclosure provides for a medical instrument with an improved attachment of an identification tag. This effect is achieved, at least in part, by the present disclosure.

Accordingly, a method for attaching an identification tag to a medical instrument is provided. The medical instrument may be a surgical instrument, such as a scissor, a scalpel, a forceps etc. The medical instrument has a first part comprising an attachment section and a second part comprising one or more functional sections. The method comprises: positioning the identification tag within the attachment section or in contact with a cover material; applying the cover material to form a tube body; and shrinking the cover material.

The tube body has at least two openings including a first opening and a second opening. Applying the cover material comprises applying the cover material such that the cover material encloses the identification tag and the attachment section, and such that the first part extends through the first opening and the second opening.

Shrinking the cover material comprises shrinking the cover material such that the first opening and the second opening are occluded by the first part.

Also disclosed is a medical instrument. The medical instrument may be a surgical instrument, such as a scissor, a scalpel, a forceps etc. The medical instrument has a first part comprising an attachment section and a second part comprising one or more functional sections. The medical instrument comprises an identification tag and a cover material.

The identification tag is positioned within the attachment section.

The cover material forms a tube body having at least two openings including a first opening and a second opening. The cover material encloses the identification tag and the attachment section, and the first part extends through the first opening and the second opening. The first opening and the second opening are occluded by the first part.

Also disclosed is a medical instrument. The medical instrument may be a surgical instrument, such as a scissor, a scalpel, a forceps etc. The medical instrument has a first part comprising an attachment section and a second part comprising one or more functional sections. The medical instrument comprises an identification tag and a cover material.

The identification tag is positioned within the attachment section.

The cover material encloses the identification tag. The cover material comprising a first fastening section fastened to the attachment section. The cover material comprising a second fastening section fastened to the attachment section.

It is envisaged that any embodiments or elements as described in connection with any one aspect may be used with any other aspects or embodiments, mutatis mutandis.

The first part of the medical instrument may be a part of the medical instrument suitable for attaching the identification tag, e.g. a part of the handle of a scalpel, or a shank of a scissor or a clamp.

The second part of the medical instrument may comprise one or more functional sections, e.g. the blade of a scalpel or a scissor. The second part may be non-suitable for attaching the identification tag.

The attachment section of the first part may designate the section of the first part enclosed by the cover material after applying the cover material. The identification tag may be positioned, or to be positioned, within the attachment section.

The first part may occlude the first opening and the second opening. Occlusion of the first opening and the second opening may mean that the cover material provides an enclosed space enclosing the identification tag and the attachment section, and that there is no opening between the outside and the inside of the enclosure through the first opening and/or the second opening. The first part occluding the first opening and the second opening may mean that the first part blocks the first opening and the second opening and/or the first part fills the first opening and the second opening.

The first part occluding the first opening and the second opening may ensure that material, such as microorganisms, germs or grime, is not trapped and concealed in an opening, thereby opposing adequate cleaning of the medical instrument. The first part occluding the first opening and the second opening may provide for adequate cleaning of the medical instrument.

The identification tag may be configured to be read by use of an electromagnetic field, e.g. by radio wave emission. For example, an electromagnetic field having a frequency between 120 KHz and 928 MHz, and/or within the Ultra High Frequency (UHF) band. For example, the frequency may be 433 MHz.

The identification tag may be an RFID tag, such as a passive RFID tag. Alternatively or additionally, the identification tag may be an NFC tag, such as a passive NFC tag.

The identification tag may be a solid identification tag, such as a solid RFID tag. Alternatively, the identification tag may be a flexible identification tag, such as a flexible RFID tag.

It is an advantage of the present disclosure that it enhance fastening of identification tag and the cover material to the medical instrument. Furthermore, the present disclosure provides that attachment of an identification tag may be more easily reversed, such as for example compared to fastening by glue, such as epoxy.

It is a further advantage of the present disclosure that attachment of an identification tag to a medical instrument is provided by a method which combines attachment of the identification tag and furthermore provides a cover for the identification tag covering for example undue protrusions or sharp edges. Furthermore, the disclosed attachment provides for adequate cleaning of the medical instrument, and provides protection of the identification tag from outside effects.

It is a further advantage of the present disclosure that covering of the identification tag may be provided. Especially in covering edges of the identification tag, the disclosure provides for a cover, which, for example, is superior to glue which tends to flow away from edges before hardening. Thus, the present disclosure provides for a method for attaching an identification tag which may be faster, easier and/or reduce costs. Thus, the present disclosure also provides a medical instrument comprising an identification tag, which may be cheaper.

It is a further advantage of the present disclosure that the attachment of an identification tag to a medical instrument may be provided to surfaces of the medical instrument, such as round surfaces, complicating attachment of the identification tag by conventional methods e.g. by fastening the identification tag by glue. For example, the disclosure provides for attachment of an identification tag to a surface having a surface area which is smaller than a surface area of the identification tag. For example, if the medical instrument is a micro instrument. Furthermore, the disclosure provides for attachment of an identification tag to a flexible surface, such as rubber, or a rubber-like material. Furthermore, the disclosure is suitable for attachment of an identification tag, wherein the identification tag is a flexible identification tag.

The identification tag may have a first surface. The identification tag may have a second surface. The second surface may be opposite the first surface, e.g. the second surface may be the back side of the first surface. The second surface may be positioned in contact with the attachment section, and/or the identification tag may be positioned such that the second surface is facing towards the attachment section. The first surface may be in contact with the cover material, and or the identification tag may be positioned such that the first surface is facing towards the cover material.

The identification tag may be fastened to the attachment section by the cover material. For example, the identification tag may be fastened to the attachment section by the cover material without an adhesive between the identification tag and the attachment section. Alternatively or additionally, an adhesive, e.g. a first adhesive, such as an adhesive substance, such as a glue, such as an epoxy, may be applied between the identification tag, such as the second surface of the identification tag, and the attachment section, e.g. to fasten the identification tag to the attachment section. The method may comprise applying an adhesive, such as the first adhesive, between the identification tag, such as the second surface of the identification tag, and the attachment section. For example, positioning the identification tag within the attachment section may comprise applying an adhesive, such as the first adhesive, between the identification tag, such as the second surface of the identification tag, and the attachment section.

The cover material may be in contact with at least a first surface of the identification tag, such as the first surface of the identification tag. The cover material may be in contact with a plurality of surfaces of the identification tag, such as the first surface and a third surface and/or a fourth surface of the identification tag.

An adhesive, such as a second adhesive, such as an adhesive substance, such as a glue, such as an epoxy, may be applied between the identification tag and the cover material, such as between the first surface and the cover material, e.g. to cover or level grooves and edges between the identification tag and the cover material. The method may comprise applying an adhesive, such as the second adhesive between the identification tag, such as the first surface of the identification tag, and the cover material. For example, applying the cover material may comprise applying an adhesive between the identification tag and the cover material.

The first and the second adhesive may be the same adhesive.

The cover material may be a material suitable for a medical instrument. For example, the cover material may be configured to withstand a sterilization procedure, such as autoclaving, e.g. the cover material may be thermostable, e.g. the cover material may be thermostable after attachment of the identification tag to the medical instrument. Alternatively or additionally, the cover material may be suitable for being present in an environment for surgical procedures. For example, the cover material may be ensured not to leak certain chemical substances and/or ensured to comply with regulatory standards.

The cover material may protect the identification tag, such as protect the identification tag from corroding. For example, the cover material may be impermeable to water.

The cover material may comprise a shrinkable material and/or a shrunk shrinkable material, such as a shrinkable material after it has been shrunken, i.e. the cover material may be formed from a shrinkable material. The cover material may be constituted of a shrinkable material and/or a shrunk shrinkable material. The cover material may be a shrinkable material and/or a shrunk shrinkable material.

The shrinkable material may, for example, be a heat shrinkable material, such as a material which shrinks upon heating. The method may comprise applying heat to the cover material. For example, shrinking the cover material may comprise applying heat to the cover material. Alternatively or additionally, the shrinkable material may be a material which shrinks upon exposure to a predefined substance, such as a chemical substance. The method may comprise applying a predefined substance to the cover material. For example, shrinking the cover material may comprise applying a predefined substance to the cover material.

The cover material may be a flexible material. For example, the cover material may comprise an elastomeric material. The cover material being a flexible material may provide that the cover material is shrinkable. Alternatively or additionally, the cover material may be a flexible material after being shrunk. The cover material being a flexible material may, for example, provide for attachment of the identification tag to a flexible material, such as rubber, or a rubber-like material.

The cover material may be a tube formed material. For example, the cover material may be a tube formed material which may have been pulled onto the attachment section to form the tube body. The method may comprise pulling the cover material onto the attachment section to form the tube body. For example, applying the cover material may comprise pulling the cover material onto the attachment section to form the tube body.

The cover material may be a sheet like material. For example, the cover material may be a sheet like material which may have been bend around the attachment section to form the tube body. The method may comprise bending the cover material around the attachment section to form the tube body. For example, applying the cover material may comprise bending the cover material around the attachment section to form the tube body.

The cover material may be a sheet like material. For example, the cover material may be a sheet like material which may have been bend around the first part to form the tube body and subsequently pulled onto the attachment section. The method may comprise bending the cover material around the first part to form the tube body and pulling the cover material onto the attachment section. For example, applying the cover material may comprise bending the cover material around the first section to form the tube body and pulling the cover material onto the attachment section.

The cover material may be a sheet like material which is pre-shaped to form a tube body. For example, the cover material may be a sheet like material which is pre-shaped to form a tube body, and which may comprise a longitudinal slit, such that an element, e.g. the first part, may be inserted into the tube body through the longitudinal slit.

The cover material, such as the cover material being a sheet like material, may comprise a first fastening section. The cover material, such as the cover material being a sheet like material, may comprise a second fastening section. The first fastening section and/or the second fastening section may be configured for fastening the cover material to the attachment section. Alternatively or additionally, the first fastening section may be configured to be fastened to the second fastening section and/or the second fastening section may be configured to be fastened to the first fastening section. The first fastening section may be configured to overlap the second fastening section and/or the second fastening section may be configured to overlap the first fastening section. For example, the cover material may be bend to form the tube body, and the first fastening section and the second fastening may be fastened together, such as fused together, such as glued together, such that the cover material forms the tube body.

The cover material may be a band like material, e.g. the cover material may be applied from a roll. The cover material may be applied by a plurality of revolutions around the attachment section to form the tube body.

Positioning the identification tag in contact with the cover material may comprise embedding the identification tag within the cover material. The identification tag may be embedded within the cover material. For example, the identification tag may be embedded within the cover material prior to applying the cover material to the medical instrument, such as the first part of the medical instrument.

The cover material may comprise cover material layers, such as two cover material layers, such as at least two cover material layers. Embedding the identification tag within the cover material may comprise embedding the identification tag between the cover material layers. The identification tag may be embedded between the cover material layers. The cover material layers may be fixed together. For example, embedding the identification tag within the cover material may comprise fixing the cover material layers together. The cover material layers may be fixed together, e.g. by gluing and/or heating.

### BRIEF DESCRIPTION OF THE DRAWINGS

The above and other features and advantages of the present invention will become readily apparent to those skilled in the art by the following detailed description of exemplary embodiments thereof with reference to the attached drawings, in which:

FIG. 1 schematically illustrates an exemplary medical instrument,

FIG. 2 schematically illustrates the exemplary medical instrument,

FIG. 3 schematically illustrates a part of an exemplary medical instrument,

FIG. 4 shows steps of an exemplary method for attaching an identification tag,

FIG. 5 schematically illustrates a part of a medical instrument,

FIG. 6 schematically illustrates a part of a medical instrument,

FIG. 7 shows steps of an exemplary method for attaching an identification tag,

FIG. 8 schematically illustrates an exemplary cover material,

FIG. 9 schematically illustrates an exemplary cover material,

FIG. 10 shows a flow diagram of an exemplary method for attaching an identification tag,

FIG. 11 shows steps of an exemplary method for attaching an identification tag, and

FIG. 12 shows steps of an exemplary method for attaching an identification tag.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

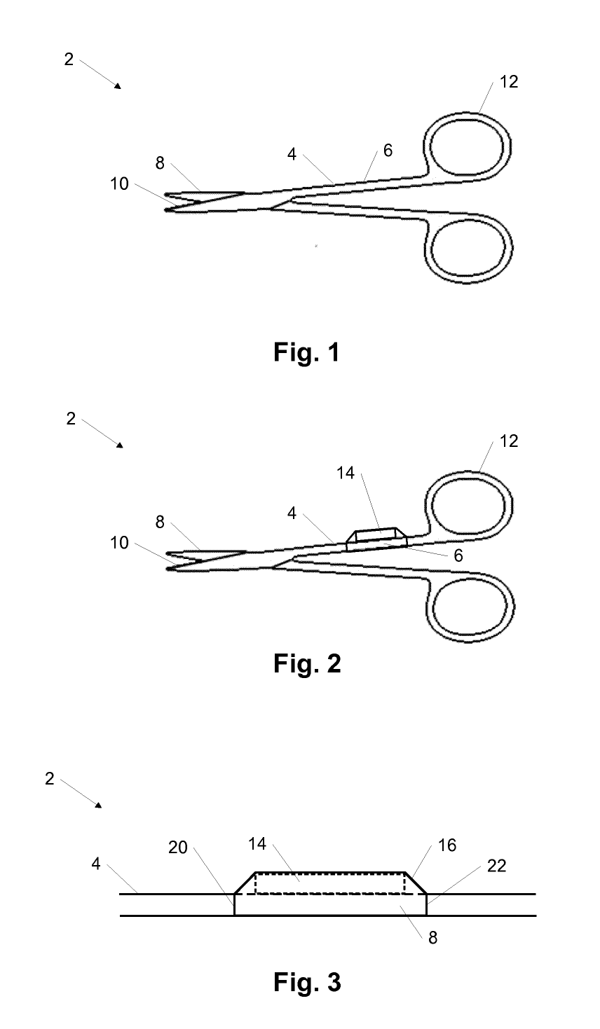

FIG. 1 schematically illustrates an exemplary medical instrument 2. In the depicted example, the medical instrument 2 is a surgical instrument. In the depicted example, the medical instrument 2 is a scissor. However, the medical instrument 2 may just as well be another medical instrument, such as a scalpel, a forceps, etc.

The medical instrument 2 has a first part 4 and a second part 8. The first part 4 comprises an attachment section 6, e.g. a section suitable for attaching an identification tag. The second part 8 comprises a functional section 10. The functional section 10 may be a section which is not suited for attachment of an identification tag, such as the blade of the scissor in the depicted example. The medical instrument may comprise additional parts, e.g. a third part 12, which may comprise another functional section, such as the finger holes, which may not be suited for attachment of an identification tag.

FIG. 2 schematically illustrates the exemplary medical instrument 2 comprising an attached identification tag 14. The identification tag 14 is positioned within the attachment section 6 of the first part 4. FIG. 3 illustrates a closer view of the attachment between the identification tag 14 and the first part 6.

FIG. 3 schematically illustrates a part of an exemplary medical instrument 2 comprising an attached identification tag 14. The medical instrument 2 has a first part 4. The first part 4 comprises an attachment section 6. The attachment section 6 is suitable for attaching the identification tag 14, e.g. the identification tag 14 does not oppose intended use of the medical instrument 2 by being positioned and/or fastened within the attachment section 6 and/or on the first part 4. The identification tag 14 is positioned within the attachment section 6.

A cover material 16 forms a tube body having at least two openings 20, 22. The two openings include a first opening 20 and a second opening 22. The cover material 16 encloses the identification tag 14 and the attachment section 6. The first part 4 extends through the first opening 20 and the second opening 22. Furthermore, the first opening 20 and the second opening 22 are occluded by the first part 4.

The cover material 16 may be a material which is shrunk after it is applied over the identification tag 14 and the attachment section 6. The cover material 16 may for example be, or have been, a shrinkable material. For example, the cover material may have been shrunk such that the first part 4 occludes the first opening 20 and the second opening 22. For example, the first opening 20 and/or the second opening 22 may have been contracted, such that the first opening 20 and the second opening 22 are occluded by the first part 4.

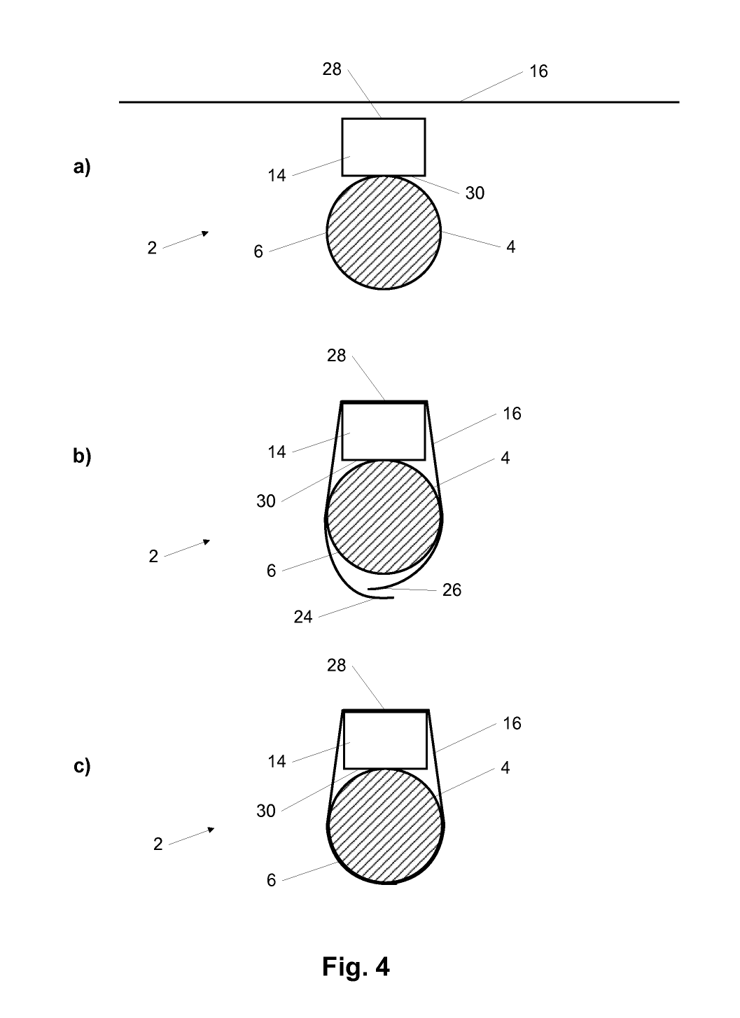

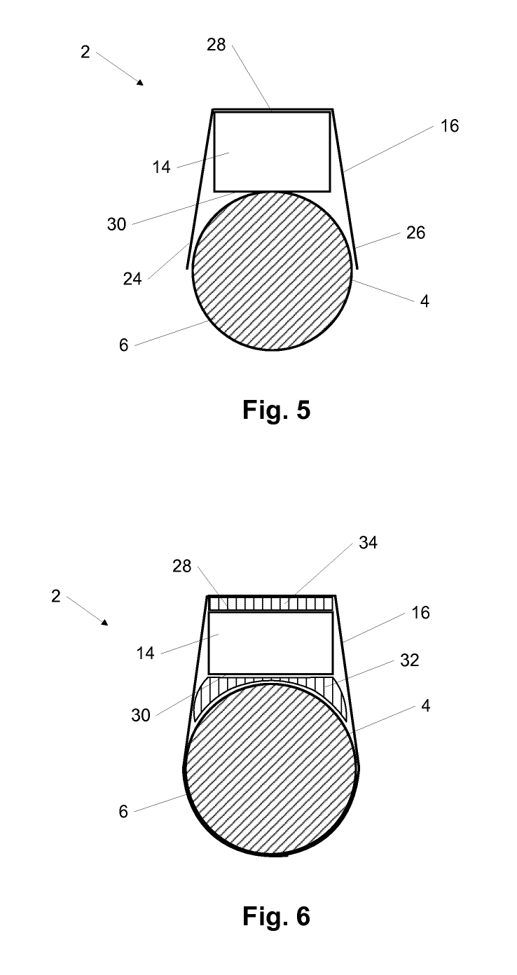

FIG. 4a-c shows steps of an exemplary method for attaching an identification tag 14 to an exemplary medical instrument 2. The medical instrument 2 comprises a first part 4. The first part comprises an attachment section 6 for attaching the identification tag 14.

FIG. 4a shows that the identification tag 14 is positioned within the attachment section 6, and a cover material 16 is applied over the identification tag 14. A first surface 28 of the identification tag 14 is facing towards the cover material. A second surface 30 of the identification tag 14 is in contact with the attachment section 6. Alternatively, the identification tag 14, such as the first surface 28 of the identification tag 14, may be positioned in contact with the cover material 16 before positioning the identification tag 14, such as the second surface 30 of the identification tag 14, in contact with the attachment section 6.

FIG. 4b shows that the cover material 16 is applied to form a tube body enclosing the identification tag 14 and the attachment section 6. The tube body is formed by bending the cover material 16 around the attachment section 6. The cover material 16 comprises a first fastening section 24 and a second fastening section 26, which is configured to fasten the cover material 16. The first fastening section 24 is configured to overlap the second fastening section 26. The first fastening section 24 and the second fastening section 26 are configured to be fastened together, e.g. by adhesive, heat treatment, chemical treatment. Thereby the cover material 16 forms a tube body enclosing the identification tag 14 and the attachment section 6.

The cover material 16 may be formed into a tube body by bending the cover material around any section of the first part 4, e.g. the attachment section 6 or another section. In case the cover material 16 is formed into the tube body by bending the cover material around a section different from the attachment section, the tube body formed by the cover material 16 may thereafter be pulled over the attachment section 6 to enclose the attachment section 6 and the identification tag 14.

FIG. 4c shows that the first fastening section 24 is fastened with the second fastening section 26, and the tube body, formed by the cover material 16, encloses the identification tag 14 and the attachment section 6. Furthermore, the cover material 16 is shrunk to tighten the cover material 16 around the attachment section 6 and the identification tag 14. Shrinking the cover material 16 may be achieved by the cover material 16 being a shrinkable material, such as a heat shrinkable material, such as a material which shrinks upon application of heat. Thus, for example, the cover material 16 may be shrunk by applying heat. Shrinking the cover material 16 also provides for the first part 4 to occlude the first opening and the second opening of the tube body (see e.g. FIG. 3). Shrinking the cover material 16 may provide that the first surface of the identification tag 14 is in contact with the cover material 16

FIG. 5 schematically illustrates a part of a medical instrument 2 with attachment of an identification tag 14 to a first part 4 comprising an attachment section 6. The first fastening section 24 is fastened to the attachment section 6, e.g. by an adhesive. The second fastening section 26 is fastened to the attachment section 6, e.g. by an adhesive. For illustrative purposes the first fastening section 24 and the second fastening section 26 are shown spaced from the attachment section 6. The first fastening section 24 may be in contact with the attachment section 6, and/or the second fastening section 26 may be in contact with the attachment section 6.

After the first fastening section 24 and the second fastening section 26 are fastened to the attachment section 6, the cover material 16 may be shrunk to tighten the cover material around the attachment section 6 and the identification tag 14.

FIG. 6 schematically illustrates a part of a medical instrument 2 with attachment of an identification tag 14 to a first part 4 comprising an attachment section 6. A first adhesive 32 is positioned between the identification tag 14 and the attachment section 6, such as between the second surface 30 of the identification tag 14 and the attachment section 6. The first adhesive 32 may be an adhesive substance, such as glue, such as an epoxy. A second adhesive substance 34 is positioned between the identification tag 14 and the cover material 16, such as between the first surface 28 of the identification tag 14 and the cover material 16. The second adhesive 34 may be an adhesive substance, such as glue, such as an epoxy. While FIG. 6 shows both a first adhesive 32 and a second adhesive 34, in other exemplary medical instruments, the first adhesive 32 may be present without the second adhesive 34, or the second adhesive 34 may be present without the first adhesive 32.

As illustrated the first adhesive 32 may be allowed to fill some or all of the space between the attachment section 6 and the identification tag 14. For example, the first adhesive 32 may provide a larger base for fastening the identification tag 14. Thus, the first adhesive 32 may stabilize the fastening of the identification tag 14 to the attachment section 6.

Additionally or alternatively, a third adhesive (not shown) may be applied on top of the cover material 16, e.g. to provide for a cover of the identification tag with an increased strength. Such third adhesive may be applied with or without the presence of the first adhesive 32 and/or the second adhesive 34.

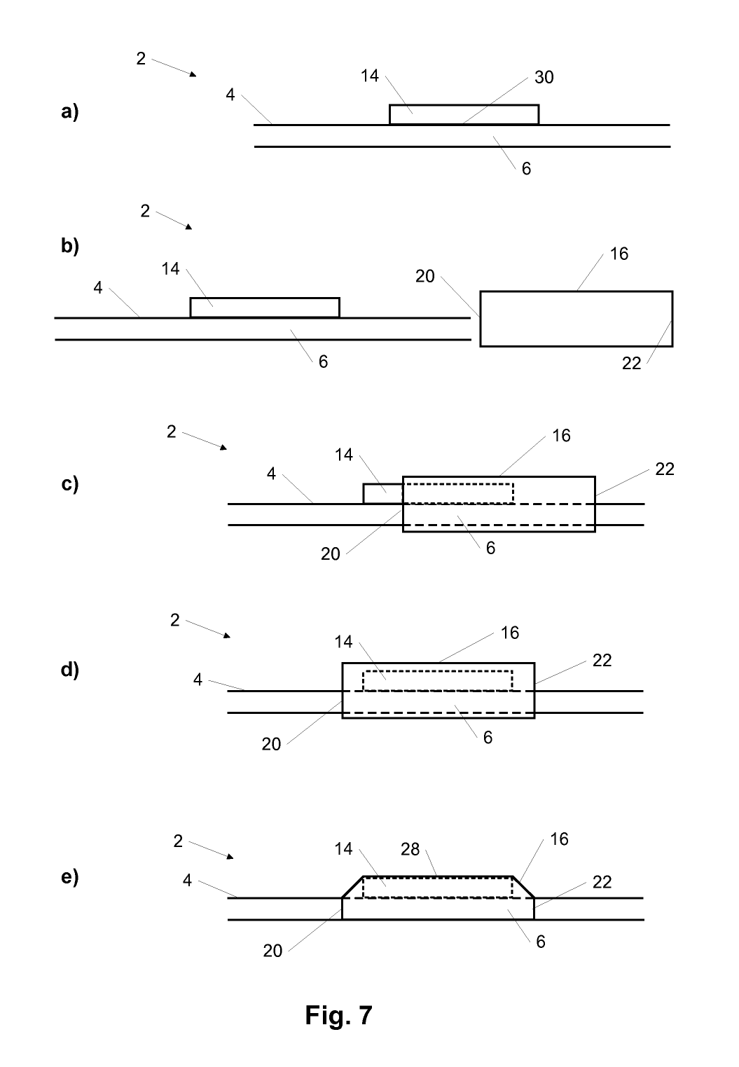

FIG. 7a-e shows steps of an exemplary method for attaching an identification tag 14 to an exemplary medical instrument 2. The medical instrument 2 comprises a first part 4. The first part comprises an attachment section 6 for attaching the identification tag 14.

FIG. 7a shows that the identification tag 14 is positioned within the attachment section 6 of the first part 4 of the medical instrument 2. A second surface 30 of the identification tag 14 is in contact with the attachment section 6.

FIG. 7b shows that a cover material 16 formed as a tube body having a first opening 20 and a second opening 22 is applied to the first part 4. In the depicted example, the cover material 16 is formed as a tube body before applying to the first part 4.

FIG. 7c shows that the cover material 16 is pulled along the first part 4. The cover material 16 is pulled onto the attachment section 6. The attachment section 6 and the identification tag enter into the tube body formed by the cover material 16 through the first opening 20.

FIG. 7d shows that the cover material 16 has been pulled onto the attachment section 6, such that the cover material 16 enclosed the attachment section 6 and the identification tag 14. The first part 4 extends through the first opening 20 and the second opening 22.

FIG. 7e shows that the cover material 16 has been shrunk to tighten the cover material 16 around the attachment section 6 and the identification tag 14. Shrinking the cover material 16 may be achieved by the cover material 16 being a shrinkable material, such as a heat shrinkable material, such as a material which shrinks upon application of heat. Thus, for example, the cover material 16 may be shrunk by applying heat. Shrinking the cover material 16 also provides for the first part 4 to occlude the first opening 20 and the second opening 22 of the tube body. Shrinking the cover material 16 may provide that a first surface 28 of the identification tag 14 is in contact with the cover material 16.

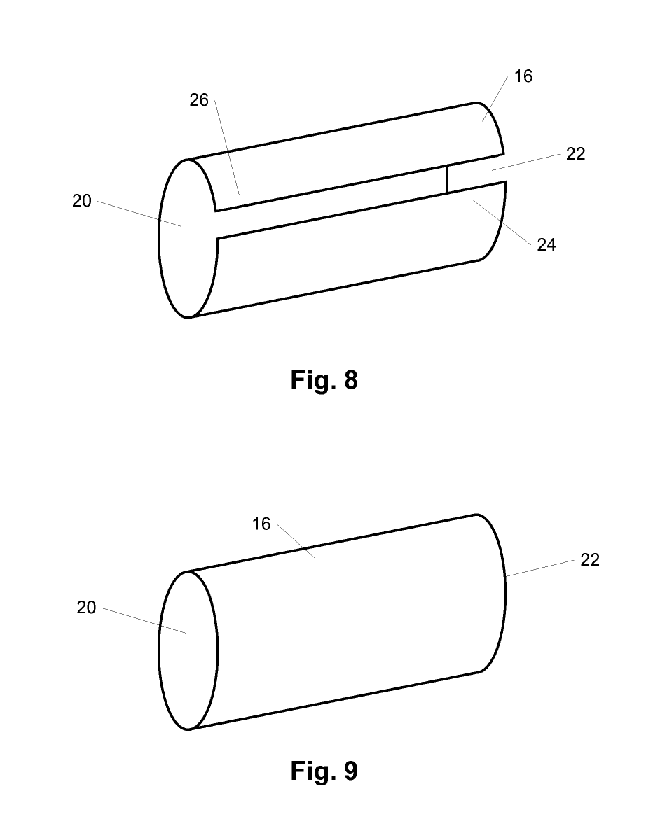

FIG. 8 schematically illustrates an exemplary cover material 16. For example a cover material 16 suitable for the example described in relation to any of FIGS. 4-6. The cover material 16 is a sheet like material, e.g. a sheet like material formed as a tube having a first opening 20 and a second opening 22. The cover material comprises a first fastening section 24 and a second fastening section 26.

The first fastening section 24 may be configured to overlap the second fastening section 26. The first fastening section 24 and the second fastening section 26 may be configured to be fastened together, e.g. by adhesive, heat treatment, chemical treatment. For example, as described in relation to FIG. 4.

Alternatively or additionally, the first fastening section 24 and/or the second fastening section 26, may be configured to be fastened to the attachment section 6, e.g. by an adhesive. For example, as described in relation to FIG. 5.

The cover material 16 may be a sheet like material which is pre-shaped into the shown tubular form, i.e. the cover material 16 may bend into the tubular form. Alternatively the cover material 16 may be bend to attain the tubular form. The cover material 16 may be a flexible material.

FIG. 9 schematically illustrates an exemplary cover material 16. For example a cover material 16 suitable for the example described in relation to FIG. 7. The cover material 16 is a tube formed material having a first opening 20 and a second opening 22. The cover material 16 may be pulled onto the attachment section as described in relation to FIG. 7. The cover material 16 may be a flexible material.

The sheet like cover material as illustrated in FIG. 8 may be advantageous if the first part comprising the attachment section is connected to parts comprising sections with a significantly larger cross sectional area, e.g. the finger rings of a scissor as illustrated in FIGS. 1-3.

The tube formed cover material as illustrated in FIG. 9 dispense the need for fastening sections, such as the first fastening section and/or the second fastening section. Thus, the tube formed cover material may have a higher strength than the sheet like cover material. Therefore, the tube formed cover material may be advantageous if it is possible to pull a tube formed cover material onto the attachment section.

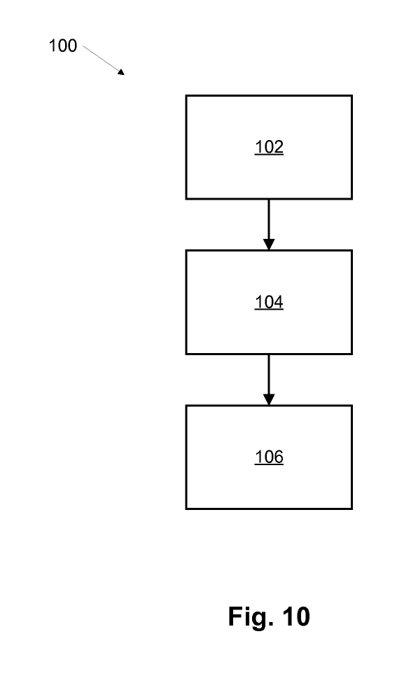

FIG. 10 shows a flow diagram of an exemplary method 100 for attaching an identification tag to a medical instrument. The medical instrument may be a surgical instrument, such as a scissor, a scalpel, a forceps etc. The medical instrument has a first part comprising an attachment section. The first part is suitable for attachment of the identification tag, e.g. the attachment section is suitable for attachment of the identification tag. The medical instrument has a second part comprising one or more functional sections, such as a blade or similar. The second part may be unsuitable for attachment of the identification tag.

The method 100 comprises positioning 102 the identification tag within the attachment section or in contact with a cover material. The identification tag may be embedded within the cover material.

The method 100 comprises applying 104 the cover material to form a tube body having at least two openings including a first opening and a second opening, such that the cover material encloses the identification tag and the attachment section, and the first part extends through the first opening and the second opening. Applying 104 the cover material is performed subsequently to positioning 102 the identification tag, either within the attachment section or in contact with the cover material.

After applying 104 the cover material, the identification tag will be within the attachment section and in contact with the cover material. Thus, regardless of, if the identification tag is positioned 102 within the attachment section before applying 104 the cover material, or if the identification tag is positioned 102 in contact with the cover material before applying 104 the cover material, the identification tag will, after applying 104 the cover material, be within the attachment section and in contact with the cover material.

The method 100 comprises shrinking 106 the cover material, such that the first opening and the second opening are occluded by the first part. Shrinking 106 the cover material may, for example, comprise applying heat to the cover material. For example, the cover material may be a heat shrinkable material, and shrinking 106 the cover material may comprise applying heat to the cover material. Shrinking 106 the cover material may provide that a first surface of the identification tag comes into contact with the cover material. Shrinking 106 the cover material may tighten the cover material around the attachment section and the identification tag.

Applying 104 the cover material may comprise bending the cover material around the attachment section to form the tube body. For example, the cover material may be a sheet like material, as described in relation to FIG. 8, and applying 104 the cover material may comprise bending the cover material around the attachment section.

Alternatively or additionally, applying 104 the cover material may comprise pulling the cover material onto the attachment section to form the tube body. For example, the cover material may be a tube formed material, as described in relation to FIG. 9, and applying 104 the cover material may comprise pulling the cover material onto the attachment section.

Alternatively or additionally, applying 104 the cover material may comprise both bending the cover material to form the tube body and pulling the cover material onto the attachment section. For example, the cover material may be a sheet like material and applying 104 the cover material may be comprise bending the cover material around the first part of the medical instrument to form the tube body and pulling the cover material onto the attachment section.

An adhesive, such as an adhesive substance, such as a glue, such as an epoxy, may be applied between the identification tag and the attachment section. For example, positioning the identification tag 102 within the attachment section may comprise applying the adhesive between the identification tag and the attachment section. Alternatively or additionally, applying 104 the cover material may comprise applying the adhesive between the identification tag and the attachment section.

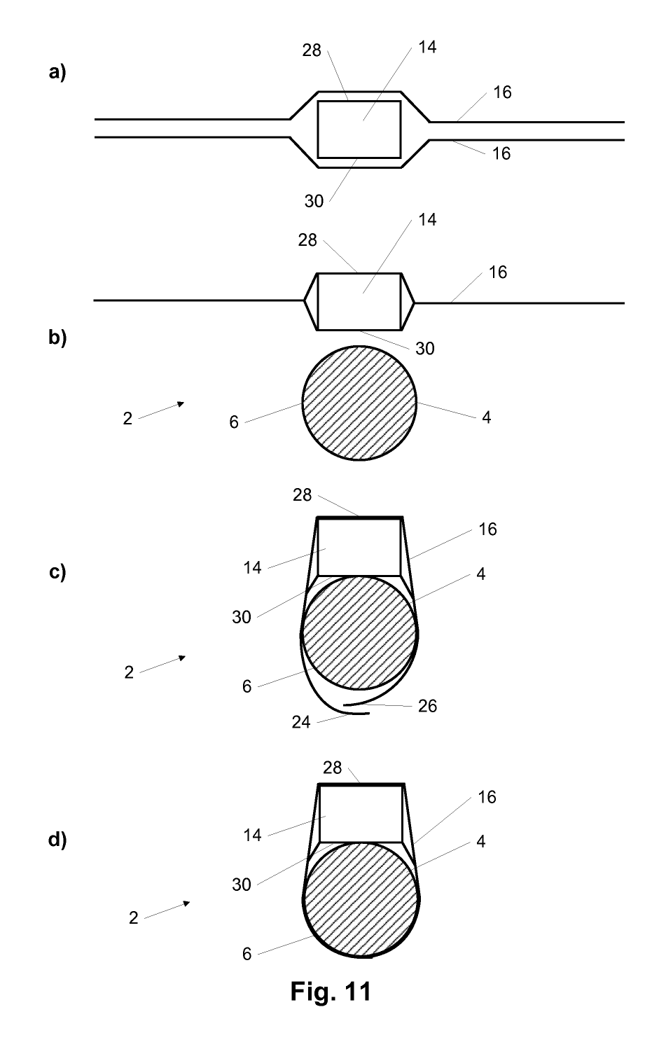

FIG. 11a-d shows steps of an exemplary method for attaching an identification tag 14 to an exemplary medical instrument 2. The medical instrument 2 comprises a first part 4. The first part comprises an attachment section 6 for attaching the identification tag 14.

FIG. 11a shows that the identification tag 14 is being embedded within the cover material 16. For example, the identification tag 14 may be embedded between two cover material layers of the cover material 16. The two cover material layers of the cover material 16 may be fixed together, e.g. glued, such as to form the cover material 16. A first surface 28 of the identification tag 14 and a second surface 30 of the identification tag 14 are contacting the cover material.

For illustrative purposes, the cover material 16 is shown providing a space on each sides of the identification tag 14. However, the cover material 16 may be in contact with all surfaces of the identification tag 14.

FIG. 11b shows that the identification tag 14 is embedded within the cover material 16. FIG. 11b furthermore shows that the identification tag 14 and the cover material 16 are positioned within the attachment section 6. The identification tag 14 is embedded in the cover material 16. The second surface 30 of the identification tag 14 is facing towards the attachment section 6.

FIG. 11c shows that the cover material 16 is applied to form a tube body enclosing the identification tag 14 and the attachment section 6. The tube body is formed by bending the cover material 16 around the attachment section 6. The cover material 16 comprises a first fastening section 24 and a second fastening section 26, which is configured to fasten the cover material 16. The first fastening section 24 is configured to overlap the second fastening section 26. The first fastening section 24 and the second fastening section 26 are configured to be fastened together, e.g. by adhesive, heat treatment, chemical treatment. Thereby the cover material 16 forms a tube body enclosing the identification tag 14 and the attachment section 6.

The cover material 16 may be formed into a tube body by bending the cover material around any section of the first part 4, e.g. the attachment section 6 or another section. In case the cover material 16 is formed into the tube body by bending the cover material around a section different from the attachment section, the tube body formed by the cover material 16 may thereafter be pulled over the attachment section 6 to enclose the attachment section 6.

FIG. 11d shows that the first fastening section 24 is fastened with the second fastening section 26, and the tube body, formed by the cover material 16, encloses the identification tag 14 and the attachment section 6. Furthermore, the cover material 16 is shrunk to tighten the cover material 16 around the attachment section 6. Shrinking the cover material 16 may be achieved by the cover material 16 being a shrinkable material, such as a heat shrinkable material, such as a material which shrinks upon application of heat. Thus, for example, the cover material 16 may be shrunk by applying heat. Shrinking the cover material 16 also provides for the first part 4 to occlude the first opening and the second opening of the tube body (see e.g. FIG. 3).

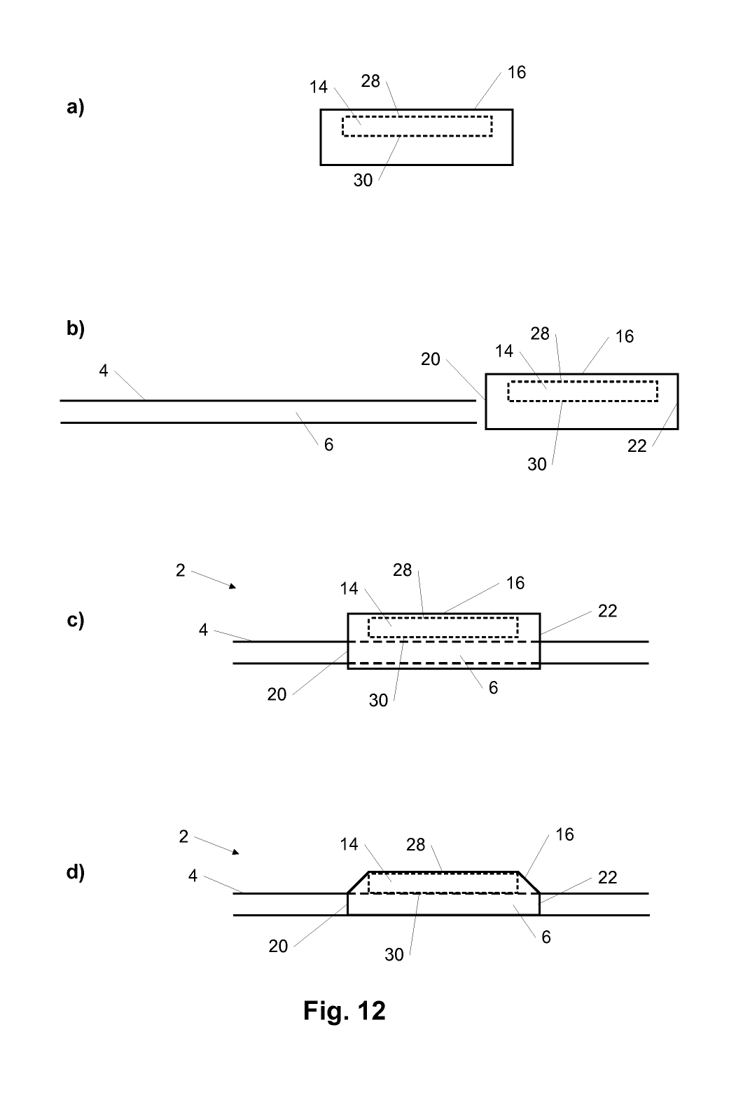

FIG. 12a-d shows steps of an exemplary method for attaching an identification tag 14 to an exemplary medical instrument 2. The medical instrument 2 comprises a first part 4. The first part comprises an attachment section 6 for attaching the identification tag 14.

FIG. 12a shows that the identification tag 14 is embedded within the cover material 16. For example, the identification tag 14 may be embedded between two cover material layers of the cover material 16 fixed together, e.g. glued, such as to form the cover material 16. A first surface 28 of the identification tag 14 and a second surface 30 of the identification tag 14 are contacting the cover material.

FIG. 12b shows that the identification tag 14 and the cover material 16 is being positioned within the attachment section 6 of the first part 4 of the medical instrument 2. The identification tag 14 is embedded in the cover material 16.

The cover material 16 is formed as a tube body having a first opening 20 and a second opening 22. In the depicted example, the cover material 16 is formed as a tube body before applying to the first part 4. Alternatively, the cover material 16 may be formed as a tube body after applying to the first part 4.

The cover material 16 and the identification tag 14 are pulled along the first part 4. The cover material 16 and the identification tag 14 are pulled onto the attachment section 6. The attachment section 6 enters into the tube body formed by the cover material 16 through the first opening 20.

FIG. 12c shows that the identification tag 14 and the cover material 16 is positioned within the attachment section 6 of the first part 4 of the medical instrument 2. FIG. 12c shows that the cover material 16 and the identification tag 14 has been pulled onto the attachment section 6, such that the cover material 16 encloses the attachment section 6 and the identification tag 14. The second surface 30 of the identification tag 14 is facing towards the attachment section 6. The first part 4 extends through the first opening 20 and the second opening 22.

FIG. 12d shows that the cover material 16 has been shrunk to tighten the cover material 16 around the attachment section 6. Shrinking the cover material 16 may be achieved by the cover material 16 being a shrinkable material, such as a heat shrinkable material, such as a material which shrinks upon application of heat. Thus, for example, the cover material 16 may be shrunk by applying heat. Shrinking the cover material 16 also provides for the first part 4 to occlude the first opening 20 and the second opening 22 of the tube body.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications and equivalents.

### LIST OF REFERENCES

2 medical instrument
4 first part
6 attachment section
8 second part
10 functional section
12 third part
14 identification tag
16 cover material
20 first opening
22 second opening
24 first fastening section
26 second fastening section
28 first surface
30 second surface
32 first adhesive
34 second adhesive
100 method for attaching an identification tag
102 positioning identification tag
104 applying cover material
106 shrinking cover material

1. A method for attaching an identification tag to a medical instrument, the method comprising:
positioning an identification tag, which comprises a first part comprising an attachment section and a second part comprising one or more functional sections, within the attachment section or in contact with a cover material;
applying the cover material to form a tube body comprising at least two openings including a first opening and a second opening, such that the cover material encloses the identification tag and the attachment section, and the first part extends through the first opening and the second opening; and
shrinking the cover material, such that the first opening and the second opening are occluded by the first part.

2-20. (canceled)

21. The method according to claim 1, wherein applying the cover material comprises bending the cover material around the attachment section to form the tube body.

22. The method according to claim 1, wherein applying the cover material comprises pulling the cover material onto the attachment section to form the tube body.

23. The method according to claim 1, wherein positioning the identification tag in contact with the cover material comprises embedding the identification tag within the cover material.

24. The method according to claim 23, wherein the cover material comprises two cover material layers and, wherein embedding the identification tag within the cover material comprises embedding the identification tag between the cover material layers and fixing the two cover material layers together.

25. The method according to claim 1, wherein positioning the identification tag within the attachment section comprises applying an adhesive between the identification tag and the attachment section.

26. The method according to claim 1, wherein shrinking the cover material comprises applying heat to the cover material.

27. A medical instrument comprising a first part, which comprises an attachment section and a second part, which comprises one or more functional sections, the medical instrument further comprising:
an identification tag positioned within the attachment section; and
a cover material forming a tube body comprising at least two openings including a first opening and a second opening, the cover material enclosing the identification tag and the attachment section, and the first part extending through the first opening and the second opening;
wherein the first opening and the second opening are occluded by the first part and, wherein the cover material is a shrunk shrinkable material.

28. The medical instrument according to claim 27, wherein the cover material is a shrunk heat shrinkable material.

29. The medical instrument according to claim 27, wherein the cover material is a sheet like material, which is bent around the attachment section to form the tube body.

30. The medical instrument according to claim 27, wherein the cover material is a tube formed material, which has been pulled onto the attachment section to form the tube body.

31. The medical instrument according to claim 27, wherein the cover material comprises an elastomeric material.

32. The medical instrument according to claim 27, wherein the cover material is in contact with at least a first surface of the identification tag.

33. The medical instrument according to claim 27, wherein the cover material is configured to withstand a sterilization procedure or autoclaving or, wherein the cover material is configured for a surgical procedure.

34. The medical instrument according to claim 27, wherein the cover material is impermeable to water.

35. The medical instrument according to claim 27, wherein the identification tag is an RFID tag, a passive RFID tag, an NFC tag, or a passive NFC tag.

36. The medical instrument according to claim 27, wherein the identification tag is configured to be read by an electromagnetic field having a frequency between 120 KHz and 928 MHz or within the Ultra High Frequency (UHF) band.

37. The medical instrument according to claim 27, wherein the identification tag is embedded within the cover material.

38. The medical instrument according to claim 37, wherein the cover material comprises two cover material layers and, wherein the identification tag is embedded between the cover material layers.

39. The medical instrument according to claim 38, wherein the cover material layers are fixed together.
